SEQUENCE LISTING (29 sequences)

SEQ ID NO: 1 - RNA, 328 nt, Mus musculus
ccuuccugagcuaccacccguacccuuucucccagcgcacacuuacugcucgauuccaggucaacaauacucacccaccccacgugcaguugcugcgaaagccaguacucacagucauggggcucauggcccuguuggauggagaacaacucuaggcagaggucucaaaggcuggggcuguguuggacagcaaucauacaguggguguccuggccagcacccaucacccugaaggcuccgcagcggccuggaguaccacaguccucaucuacacuagugaugacacccacgcacaccccaaccuacguuuccagucgguccacguuug

SEQ ID NO: 2 - RNA, 40 nt, Artificial Sequence
  antisense oligonucleotide
cacagccccagccuuugagaccucugcccagaguuguucu

SEQ ID NO: 3 - RNA, 57 nt, Artificial Sequence
  antisense oligonucleotide
gmcmcmcmcmamgmcmcmumumumgmamgmamcmcmumcugcccagaguuguucucc

SEQ ID NO: 4 - RNA, 61 nt, Artificial Sequence
  antisense oligonucleotide
gmcmcmcmcmamgmcmcmumumumgmamgmamcmcmumcmumgmcmccagaguuguucucc

SEQ ID NO: 5 - RNA, 78 nt, Artificial Sequence
  antisense oligonucleotide
cmamcmamgmcmcmcmcmamgmcmcmumumumgmamgmamcmcmumcmumgmumcmcmamgmamgmumumgmumumcm

SEQ ID NO: 6 - RNA, 39 nt, Artificial Sequence
  antisense oligonucleotide
cacagccccagccuuugagaccucuguccagaguuguuc

SEQ ID NO: 7 - DNA, 71 bp, Mus musculus
ggggctcatggccctgttggatggagaacaactctaggcagaggtctcaaaggctggggctgtgttggaca

SEQ ID NO: 8 - DNA, 23 bp, Artificial Sequence
  Primer
agtactcacagtcatggggctca

SEQ ID NO: 9 - DNA, 22 bp, Artificial Sequence
  Primer
gccaggacacccactgtatgat

SEQ ID NO: 10 - DNA, 16 bp, Artificial Sequence
  Primer
tggggctcatggccct

SEQ ID NO: 11 - DNA, 61 bp, Artificial Sequence
  A or G Construct
gttggatggagaacaactctargggcagaggtctcaargaggctggggctgtgttggacag

SEQ ID NO: 12 - DNA, 56 bp, Artificial Sequence
  Dispensation order
agctgactggagagcgagctcatagtcagagtctgcgagagctggctgtgctgaca

SEQ ID NO: 13 - DNA, 38 bp, Artificial Sequence
  dispensation order
cgtgatgagacactcgtagcagagtctgcagagctgca

SEQ ID NO: 14 - DNA, 71 bp, Artificial Sequence
  A or G construct
gttggargtggarggaacaargctctargggcarggaggtctcaaargggctggggctgtgttggargcag

SEQ ID NO: 15 - DNA, 64 bp, Artificial Sequence
  ESS of XIAP
gttgcccaggatggagtgcaatggcacaatcttggctcatggcaaactctgcctcgcaagcgga

SEQ ID NO: 16 - DNA, 51 bp, Artificial Sequence
  ESS of 5-HT2RC
tatcgctggatcggtatgtagcaatacgtaatcctattgagcatagccgtt

SEQ ID NO: 17 - RNA, 24 nt, Artificial Sequence
  24-mer AON targeting XIAP
gagccaagauugugccauugcacu

SEQ ID NO: 18 - RNA, 19 nt, Artificial Sequence
  19-mer AON targeting XIAP
ccaagauugugccauugca

SEQ ID NO: 19 - DNA, 24 bp, Artificial Sequence
  24-mer AON targeting 5-HT2RC
aggauuacguauugcuacataccg

SEQ ID NO: 20 - DNA, 19 bp, Artificial Sequence
  19-mer AON targeting 5-HT2RC
uacguauugcuacataccg

SEQ ID NO: 21 - DNA, 11 bp, Artificial Sequence
  cDNA of XIAP ESS RNA
artggcacart

SEQ ID NO: 22 - DNA, 11 bp, Artificial Sequence
  cDNA of XIAP ESS RNA
aatggcacaat

SEQ ID NO: 23 - DNA, 10 bp, Artificial Sequence
  cDNA of 5-HT2RC ESS RNA
gcartacgta

SEQ ID NO: 24 - DNA, 19 bp, Artificial Sequence
  XIAP forward PCR primer
ctactccggagcctgaggt

SEQ ID NO: 25 - DNA, 22 bp, Artificial Sequence
  XIAP reverse PCR primer
tgggagatgaatcacttgaggt

SEQ ID NO: 26 - DNA, 23 bp, Artificial Sequence
  XIAP sequencing primer
cagaaattcaagactagcctggc

SEQ ID NO: 27 - DNA, 25 bp, Artificial Sequence
  5-HT2RC forward PCR primer
ggcaatcctttatgattatgtctgg

SEQ ID NO: 28 - DNA, 23 bp, Artificial Sequence
  5-HT2RC reverse PCR primer
tgcccaaacaatagcaatcttca

SEQ ID NO: 29 - DNA, 20 bp, Artificial Sequence
  5-HT2RC sequencing primer
tcatgatggccttagtccgc

(19) United States
(12) Patent Application Publication
(10) Pub. No.: US 2023/0235322 A1
(43) Pub. Date: Jul. 27, 2023

(54) RNA EDITING INHIBITORS AND METHODS OF USE

(71) Applicant: ProQR Therapeutics II B.V., Leiden (NL)

(72) Inventors: Janne Juha Turunen, Leiden (NL); Lenka Van Sint Fiet, Leiden (NL); Lisanne Alieda Van Wissen, Leiden (NL)

(21) Appl. No.: 17/605,843

(22) PCT Filed: Apr. 9, 2020
(86) PCT No.: PCT/EP2020/060291

§ 371 Date: Oct. 22, 2021

(30) Foreign Application Priority Data
Apr. 24, 2019 (GB) 1905732.2

(51) Int. Cl. C12N 15/113

(52) U.S. Cl. CPC C12N 15/113; C12N 2310/11; C12N 2310/315; C12N 2310/321; C12N 2310/3231

(57) ABSTRACT

An antisense oligonucleotide (AON) capable of inhibiting ADAR-mediated deamination of a target adenosine present in an editing-site sequence (ESS) of a target RNA molecule, wherein under physiological conditions the ESS would hybridize with an editing-site complementary sequence (ESCS) of an RNA molecule to form a double stranded RNA complex, wherein the AON comprises a sequence configured to compete with the ESCS for hybridization with the ESS.

### RELATED APPLICATIONS

This application is a §371 National Stage Application of PCT/EP2020/060291, filed Apr. 9, 2020, which claims priority to and the benefit of United Kingdom patent application No. 1905732.2, filed Apr. 24, 2019, the entire disclosures of each of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on May 11, 2022, is named PQR-031WOUS_SL2 and is 6,692 bytes in size.

### TECHNICAL FIELD

This invention is in the field of medicine, specifically redirecting or suppressing physiological editing by endogenous RNA editing enzymes in a patient in need thereof. Specifically an antisense oligonucleotide (AON) is used to inhibit ADAR-mediated deamination of a target adenosine present in an editing-site sequence (ESS) of a target RNA molecule, by competing with an editing-site complementary sequence (ESCS) for binding to the ESS and inhibiting ADAR-mediated editing of the target adenosine.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called adenosine deaminase and cytidine deaminase, respectively. The most extensively studied RNA editing system is the adenosine deaminase enzyme.

Adenosine deaminase is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an adenosine (A) into inosine (I) in a nearby, more or less predefined, position in the target

RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result thereof, exons may be included or skipped. The adenosine deaminases are part of a family of enzymes known as Adenosine Deaminases acting on RNA (ADAR), including human deaminases hADAR1 and hADAR2, as well as hADAR3 for which no deaminase activity has been shown yet.

There are a number of medical conditions where it would be favourable to use an intervention that encourages A to I conversions. The design and use of editing oligonucleotides (EONs) that bind to a target RNA and then recruit and direct ADAR to catalyse a specific and desired A to I conversion has been described (e.g. Montiel-Gonzalez et al. PNAS 2013, 110(45):18285-18290; Vogel et al. 2014. Angewandte Chemie Int Ed 53:267-271; Woolf et al. 1995. PNAS 92:8298-8302).

There are also a number of medical conditions where it would be favourable to prevent A to I conversions that occur naturally, and the use of inhibitors to disrupt ADAR-mediated A to I conversions have also been described (e.g. Schirle et al. 2010, Org. Biomol. Chem., 8,4898-4904; Penn et al. 2013, Nucleic Acids Res. 41(2): 1113-23; Mizrahi et al. 2013, ACS Chem Biol. 8(4):832-9).

Di Giorgio et al. (2020; doi.org/10.1101/2020.03.02.973255) describe the observation of A to I and C to U RNA editing in the host cell upon entry of the SARS-CoV-2 virus, which was the cause of the global Covid-19 pandemic spreading from the province of Wuhan, China to nearly all countries around the world in the first few months of 2020, It is unknown whether the RNA editing is in fact a defence mechanism of the cell, or that the virus uses the machinery for its own benefit. Importantly, if that observed RNA editing is a mechanism that is used by the virus to enlarge its transcript repertoire, it would be favourable to inhibit such RNA editing.

Schirle et al. discloses inhibition using a small peptide that intercalates with the dsRNA helix and hinders ADAR binding and activity. One drawback is that this inhibitor only works with a specific secondary structure of the target helix, specifically sites in duplex RNA bearing a 5′-PyPu-3′ sequence flanked on each 3′ side by bulges or loops. Most clearly this is demonstrated in FIG. 5A of Schirle et al., where the inhibitor effectively inhibits editing of a construct with the loop, but much less so with a construct lacking the loop.

RNA should typically be double stranded in order for ADAR to bind and effect the A to I conversion, although perfect complementarity is not generally required as most ADAR targets are formed by double stranded structures that are interrupted by mismatches, bulges or loops of varying sizes. In any event, the double stranded RNA comprises an editing-site sequence (ESS) which comprises the adenosine that would be edited by the ADAR, and an editing-site complementary sequence (ESCS) that hybridizes with the ESS. This can occur, for example, in a stem-loop, or hairpin, structure in endogenous RNA. Mizrahi et al. and Penn et al. disclose the use of AONs that hybridize with the ESCS and the result is that the ESS (and any adenosines therein) are rendered as a single stranded RNA that the ADAR would not bind to.

The present invention provides alternative inhibitors that offer further options and improvements over known inhibitors.

### SUMMARY OF THE INVENTION

According to a first aspect, the present invention provides an antisense oligonucleotide (AON) capable of inhibiting ADAR-mediated deamination of a target adenosine present in an editing-site sequence (ESS) of a target RNA molecule, wherein under physiological conditions the ESS would hybridize with an editing-site complementary sequence (ESCS) of an RNA molecule to form a double stranded RNA complex, wherein the AON comprises a sequence configured to compete with the ESCS for hybridization with the ESS.

The inventors have shown for the first time that an AON according to the invention can compete with the ESCS for binding with the ESS. A dsRNA complex is formed between the AON and the ESS, but the AON is configured to inhibit ADAR-mediated A to I conversion. As such, the inventors have provided a method of inhibition that does not rely on the ESS being rendered single-stranded in order to prevent ADAR-mediated A to I conversion. Importantly, once the A to I conversion has taken place, under physiological conditions the reverse reaction is generally not observed. As such, the AON has been shown to bind with sufficient affinity to hinder the ESCS binding, even for brief periods, needed to enable the A to I conversion. By providing an AON that inhibits ADAR-mediated A to I conversions by targeting the ESS, particular problems of the prior art are overcome. For example, use of an oligonucleotide that binds to the ESCS can suffer from not always knowing the identity of the ESCS. The exact sequences functioning as the ESCS are not readily identifiable in many cases, and cannot be directly inferred from sequence information alone, as ADAR-mediated editing does not require full complementarity between the two strands. Interfering with the ESCS in a complex physiological environment with many RNAs suffers from the possibility that alternative sequences may start to function as the ESCS and editing still occurs. In contrast, targeting the ESS itself solves that problem. Small changes in ESCS RNA structure can also result in loss of the inhibitory effect of AONs that target the ESCS (e.g. Mizrahi et al. 2013). Targeting the ESS can also allow for lower concentrations of AON than required when targeting the ESCS. This is particularly the case where there is an excess of the ESCS, or multiple homologous ESCS sequences, such as from sequences present in repetitive elements, where larger concentrations of AON would be required to saturate the ESCS. The AON of the invention can easily be designed, since the location of the edited sites on the target strand are clearly known, and it is not strictly necessary to know which complementary sequences function as ESCS sequences (whether the ESCS is on the same RNA molecule (in cis) or on a different RNA molecule (in trans)).

The AONs of the invention therefore allow for targeted reduction of ADAR-mediated deamination. This overcomes problems of indiscriminate non-targeted reduction of ADAR-mediated deamination, such as by reduction of ADAR expression or sequestering ADARs. Such a non-targeted approach would likely lead to a greater incidence of unwanted and undesirable side effects.

In addition to the AON being able to bind to the ESS in place of the ESCS, the dsRNA duplex comprising the ESS and AON must not support deamination of the target adenosine. Therefore, the structure and/or chemical composition of the AON-RNA duplex must be such that it either completely prevents ADAR binding or, if it allows ADAR binding, does not allow the deamination reaction to proceed. This can be achieved by utilising an AON configured such that the duplex formed by the AON and ESS contains structural features (mismatches, bulges and/or loops) that prevent productive interaction of ADAR with the duplex. Another method which has been shown to result in inhibition of editing is to chemically modify the AON in such a way that it does not allow editing to take place. For example, WO2016/097212 and WO2019/158475 relate to oligonucleotides designed to recruit ADAR in order to actively enforce editing at a chosen position in a therapeutically relevant RNA. Test subjects (control AONs) therein show that alkyl modifications of the ribose 2′ position (2′-OMe and 2′-MOE) result in inhibition of editing when positioned too close to the target adenosine. Based on computational modelling, this effect is explained to be caused by the propensity of these modifications to alter the interaction with the ADAR deaminase domain. Similarly, computational modelling has been used to indicate that other modifications likewise interfere with deaminase activity when placed in certain positions relative to the target adenosine, including a ribose modification UNA (unlocked nucleic acid), as well as internucleoside linkage modifications, including phosphorothioate and phosphonoacetate linkages (e.g. WO2019/219581 and PCT/EP2020/053283 (unpublished)). In addition to, or alternative to, backbone modifications (involving the ribose moiety or the internucleosidic linkage) the nucleobase may be modified in the ESS-targeting AON. Combinations of backbone and nucleobase modifications can also be used.

These previous studies contained aspects of preventing ADAR-mediated deamination of certain adenosines, however, the purpose of the oligonucleotides was to induce editing of an adenosine. As editing had to be induced, these studies inherently did not concern RNA that was naturally edited by formation of an RNA duplex with a pre-existing ESCS. The inventors of the present invention identified that modifications that brought about prevention of ADAR function could be exploited for the purpose of inhibiting editing on desired adenosines through successfully competing with the pre-existing ESCS. In one embodiment, AONs can be uniformly modified with one type of such inhibitory modification, or with a combination of different chemical modifications.

The AON of the present invention is capable of inhibiting ADAR-mediated deamination of a target adenosine. As mentioned, there are two steps involved in this. The first is competition with the ESCS. Under physiological conditions, the ESS would hybridize with an ESCS of an RNA molecule to form a double stranded RNA complex. This double stranded RNA complex would allow for recruitment of an endogenous ADAR, and thus enable ADAR-mediated deamination of the target adenosine. The AON of the invention comprises a sequence configured to compete with the ESCS for hybridization with the ESS. This means that the AON of the present invention has a sequence that is substantially complementary to the ESS. In an embodiment, the AON is fully complementary to the ESS, but this is not a requirement for inhibition. Indeed, the AON can comprise one or more mismatches, bulges or loops and still hybridize with the ESS. The design of AONs that hybridize with sense sequences while bearing one or more mismatches, bulges or loops is known and it can be readily determined if there is sufficient complementarity for an AON to hybridize with the ESS under physiological conditions, for example by an assay to determine if there is successful reduction of ADAR-mediated deamination, implying successful hybridization. The second step is that the ESS-AON duplex must inhibit ADAR-mediated deamination of the target adenosine. This can occur through prevention of ADAR binding, for example through creation of a secondary structure, mismatch and/or bulge that is too large to fit into the recognition or deamination sites of the ADAR protein. Alternatively, if ADAR can bind to the ESS-AON duplex and the target adenosine is located within the ADAR deaminase active site, the AON should comprise residues and or modifications that inhibit successful catalytic activity of the active site upon the target adenosine, for example by a modification of the 2′-O ribosyl at an appropriate nucleotide or nucleotides on the AON.

The AON of the present invention can inhibit ADAR-mediated deamination of a target adenosine through the nucleotide in the AON opposite the target adenosine and/or the nucleotides in the AON opposite the nucleotides surrounding the target adenosine being chemically modified to compete with the ESCS and inhibit the ADAR-mediated deamination of the target adenosine. It is preferred that the chemical modification allows the nucleotide(s) to contribute to hybridization interactions with the ESS, and thus competition with the ESCS, but this is not essential. The chemical modification can have deleterious effects of hybridization interactions with the ESS, as long as it does not prevent hybridization of the overall AON with the ESS and competition with the ESCS. In a particularly preferred embodiment, the nucleotide in the AON opposite the target adenosine is chemically modified. It is also possible that one or both of the nucleotides in the AON opposite the nucleotides neighbouring the target adenosine are chemically modified instead of, or in addition to, the nucleotide in the AON opposite the target adenosine. It is envisioned that certain AONs may lack a nucleotide that is “opposite” the target adenosine. That is, the AON is (at least) one nucleotide shorter than the ESS in the region opposite the target adenosine such that hybridisation causes the target adenosine to bulge out from the dsRNA duplex. In this case, chemical modification to one or more of the nucleotides in the AON opposite the nucleotides surrounding the target adenosine is preferred.

In one embodiment, the nucleotides surrounding the target adenosine consist of the adjacent two bases in the 3′ direction and the adjacent two bases in the 5′ direction, preferably the adjacent base in the 3′ direction and the adjacent base in the 5′ direction. It is possible that all of the nucleotides in the AON that are opposite these adjacent bases are chemically modified instead of, or in addition to, the nucleotide in the AON that is opposite the target adenosine.

In one embodiment, the AON inhibits ADAR-mediated deamination of a single target adenosine. In a preferred embodiment, the AON inhibits ADAR-mediated deamination of all adenosines in the target RNA molecule that are incorporated in the AON-ESS duplex. In particular, it is preferred that the nucleotides opposite all adenosines in the target RNA molecule are each chemically modified to compete with the ESCS and inhibit the ADAR-mediated deamination of each corresponding adenosine in the target RNA molecule.

The chemical modification can comprise a 2′-O ribosyl derivative, in particular a 2′-O-alkyl derivative. Particularly preferred derivatives include 2′-O-methyl (2′-OMe), or 2′-O-methoxyethyl (2′-MOE), more preferably 2′-MOE. Other ribosyl modifications that are encompassed by the present invention are Locked Nucleic Acid riboses (2′-4′-LNA) and constrained Ethyl (cEt). Incorporation of these sugar modifications into the AON can serve to increase binding strength of the AON to the ESS and may positively influence competition with the ESCS.

In one embodiment, the nucleotide opposite the target adenosine is guanosine. Where the nucleotide opposite the target adenosine is guanosine, deamination of the target adenosine can be inhibited without chemical modification of the guanosine. Of course, it is possible to chemically modify the guanosine in pursuit of further improvements to inhibition of ADAR activity. In one preferred embodiment, the nucleotide opposite the target adenosine is uridine. When the nucleotide opposite the target adenosine is uridine, it is generally required that the uridine and/or the nucleotides in the AON opposite the nucleotides surrounding the target adenosine are chemically modified to compete with the ESCS and inhibit the ADAR-mediated deamination of the target adenosine.

For clarity, AONs according to the invention may comprise any combination of modifications, including sequence alteration (affecting complementarity with the ESS), nucleobase modification, sugar modification and internucleosidic linkage modification, in one or more positions in the AON.

The AONs of the invention are typically intended for therapeutic use. As such, the AONs are preferably for use with endogenous ADAR and endogenous target RNA molecule. It is also preferred that the ESCS is of an endogenous RNA molecule. The ADAR is preferably a naturally expressed (endogenous) eukaryotic ADAR. In particular, the ADAR is preferably ADAR1 or ADAR2, more preferably human ADAR1 or human ADAR2. In a preferred embodiment, the ESS and ESCS are located on the same RNA molecule. A common source of endogenous dsRNA that is targeted by ADAR under physiological conditions is understood to be the case where an ESS and ESCS on the same RNA molecule hybridize to form an RNA secondary structure such as a stem-loop structure. Preferably, the AON is capable of inhibiting ADAR-mediated deamination of a target adenosine present in an ESS of a target RNA molecule, wherein under physiological conditions the ESS would hybridize with an ESCS of an RNA molecule in a cell, preferably a human cell, to form a double stranded RNA complex.

The ESS is preferably of a pre-messenger RNA, a messenger RNA, a ribosomal RNA, a transfer RNA, a long non-coding RNA, or a miRNA. Preferably the target adenosine is located within a coding sequence of an RNA molecule. In this case, ADAR-mediated deamination of a target adenosine to form inosine would mean that the adenosine is instead read as a guanine, leading to the potential for incorrect translation and the potential for a functionally deficient peptide or protein product. The AONs of the invention would then help to ensure that the correct RNA sequence is maintained. It is also possible that the target adenosine may be located within a non-coding sequence of an RNA molecule, such as in a position that is critical to a secondary structure, for example that is important for correct intron excision, or in a regulator region such as a binding site for miRNAs or a protein binding site.

AONs according to the invention may reduce or prevent editing over the entire range of complementarity between AON and ESS, or a part thereof, or even for selected adenosines within the ESS region. In case reduction or prevention of editing is intended only for specific regions or adenosines, AON design should not completely prevent binding of ADAR to the AON-ESS complex.

The AON is preferably at least 10, 11, 12, 13, 14, 15, 16, 17, 18, 19, 20, 21, 22, 23, 24, 25, 26, 27, 28, 29, 30, 31, or 32 nucleotides in length, and preferably also shorter than 100 nucleotides, preferably shorter than 60 nucleotides.

According to a second aspect, the invention provides a pharmaceutical composition comprising an AON according to the first aspect of the invention, plus a pharmaceutically acceptable carrier, excipient and/or adjuvant.

According to a third aspect, the invention provides an AON according to the first aspect of the invention or a pharmaceutical composition according to the second aspect of the invention, for use in inhibiting ADAR-mediated deamination of a target adenosine present in an ESS of a target RNA molecule, wherein under physiological conditions the ESS would hybridize with an ESCS of an RNA molecule in a cell to form a double stranded RNA complex. In a preferred embodiment, the AON is for use in reduction of RNA damage (or RNA editing). The AON can be for use in the treatment or prevention of genetic disorders, particularly disorders associated with erroneous or unwanted ADAR-mediated adenosine deamination, such as treatment or prevention of cancer, metabolic disorders and/or mental disorders, preferably wherein the cancer is hepatocellular carcinoma, esophageal squamous cell carcinoma, non-small-cell lung cancer, colorectal cell carcinoma, cervical cancer, multiple myeloma, breast cancer, lung adenocarcinoma, prostate cancer, chronic myelogenous leukemia, head and neck squamous cell carcinoma, kidney renal papillary cell carcinoma, thyroid carcinoma or uterine corpus endometrial carcinoma, preferably wherein the metabolic disorder is hyperuricemia, obesity or cardiovascular disease, preferably wherein the mental disorder is depression, bipolar disorder, schizophrenia or suicide risk. In another preferred embodiment, the AON is for use in the treatment or prevention of a viral infection, more preferably a respiratory viral infection, to inhibit RNA editing caused by the virus that has entered the host cell and which employs the RNA editing machinery of the host cell for its own benefit, for instance for increased replication or for broadening its transcript repertoire, or when the virus causes the host cell to start editing its own RNA in response to the viral infection. In such a case the RNA editing may not be considered ‘erroneous’ (from the viral perspective) but would not be desired (= unwanted).

According to a fourth aspect, the invention provides a method of inhibiting ADAR-mediated deamination of a target adenosine present in an ESS of a target RNA molecule, wherein under physiological conditions the ESS would hybridize with an ESCS of an RNA molecule in a cell to form a double stranded RNA complex, the method comprising administering an effective amount of an AON according to the first aspect of the invention or a pharmaceutical composition according to a second aspect of the invention to a patient in need thereof. The method can be used for the reduction or prevention of RNA damage (or RNA editing). The method can comprise treating conditions associated with erroneous ADAR-mediated adenosine deamination, preferably treatment or prevention of (respiratory) viral infections, cancer, metabolic disorders and/or mental disorders, preferably wherein the cancer is hepatocellular carcinoma, esophageal squamous cell carcinoma, non-small-cell lung cancer, colorectal cell carcinoma, cervical cancer, multiple myeloma, breast cancer, lung adenocarcinoma, prostate cancer, chronic myelogenous leukemia, head and neck squamous cell carcinoma, kidney renal papillary cell carcinoma, thyroid carcinoma or uterine corpus endometrial carcinoma, preferably wherein the metabolic disorder is hyperuricemia, obesity or cardiovascular disease, preferably wherein the mental disorder is depression, bipolar disorder, schizophrenia or suicide risk.

With cancer, the AONs according to the invention can be used to prevent A to I mutations in AZIN1 (preferably which result in the S367G mutation; HCC/ESCC/CRC/BC/NSCLS), BLCAP (preferably with result in mutations at sites within the YXXQ motif; CC/HCC), GLI1 (preferably which result in the R701G mutation; MM), NEIL (MM), DHFR (BC), FAK (LUAD), PCA3 (PC), pri-let-7d (CML), LIN28 (CML), miR-200b-3p (CML/HNSC/KIRP/THCA/UCEC), HNRPLL (KC/BLC), SLC22A3 (preferably which result in the N72D mutation; GC/ESCC), wherein the acronyms represent: HCC: hepatocellular carcinoma; ESCC: esophageal squamous cell carcinoma; CRC: colorectal cancer; BC: breast cancer; NSCLS: non-small-cell lung cancer; CC: cervical cancer; MM: multiple myeloma; LUAD: lung adenocarcinoma; PC: prostate cancer; CML: chronic myelogenous leukemia; HNSC: head and neck squamous cell carcinoma; KIRP: kidney renal papillary cell carcinoma; THCA: thyroid carcinoma; UCEC: uterine corpus endometrial carcinoma; KC: kidney cancer; BLC: bladder cancer; GC: gastric cancer. Further detail on these A to I mutations and the associated conditions are described in Kung et al., 2018 (“The Role of RNA Editing in Cancer Development and Metabolic Disorders”, Frontiers in Endocrinology, 9, Article 762, 1-27) and in Xu et al., 2019 (“ADAR1 Editing and its Role in Cancer”, Genes, 10:12, 1-12).

With metabolic disorders, the AONs according to the invention can be used to prevent A to I mutations in GluR-B (Q607R), miR376, 5-HT2cR, rhythmic genes or let-7. Further detail is set out in Kung et al. 2018.

With mental disorders, the AONs according to the invention can be used to prevent A to I mutations in 5-HT2cR (preferably A to I mutations which mutate Ile156, Asn158 and/or Ile160). Further detail is set out in Dracheva et al., 2008 (“Increased serotonin 2C receptor mRNA editing: a possible risk factor for suicide”, Molecular Psychiatry, 13, 1001-1010).

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows the 5′ to 3′ sequence of the Idua RNA target strand (ESS; SEQ ID NO: 1), where the target adenosines in the target strand codons W392X and A393 used in the examples are shown in bold. Also shown are the sequences of a complementary RNA guide strand (ESCS; IDUA7; SEQ ID NO: 2) and four AONs: IDUA31 (SEQ ID NO: 3), IDUA32 (SEQ ID NO: 4), ADAR102-20 (SEQ ID NO: 5) and ADAR102-32 (SEQ ID NO: 6). Uppercase letters indicate unmodified RNA, lowercase letters indicate 2′-OMe modified nucleotides, underlined lowercase letters indicate 2′-MOE modified nucleotides, and phosphorothioate (PS) linkages are indicated by asterisks.

FIG. 2 shows (top) a comparison of the complementarity of the edited strand (here given as DNA; SEQ ID NO: 7) and the RNA guide strand (IDUA7; SEQ ID NO: 2), and the partially 2′-OMe-modified AONs used in Example 2 (middle and bottom). A partial sequence of the edited-site (target) RNA strand from the mouse Idua (W392X) mRNA is shown 5′-3′ (here given as DNA; SEQ ID NO: 7), with the complementary sequence of AONs IDUA31 (SEQ ID NO: 3) and IDUA32 (SEQ ID NO: 4) below it in the 3′-5′ direction. Unmodified RNA nucleotides are indicated in capital letters, and 2′-OMe modified positions in lowercase letters. The one mismatched position is indicated by the C which is displaced from the rest of the sequence. The target adenosine (in codon A393) for Example 2 is highlighted in bold, underlined and with an arrow.

FIG. 3 is a chart showing the suppression of editing when the target RNA comprising the ESS is duplexed with AONs containing chemical modifications. A duplex between Idua W392X RNA (the ESS) and an RNA guide (the ESCS; IDUA7) was used as a positive control. The results of including the AONs ADAR102-20 and ADAR 102-32 with the target RNA (in the absence of the ESCS, i.e. without analysing effects of competition at this stage) are also shown. Results are shown as the percentage of edited nucleotide (i.e. nucleotides read as G in the pyrosequencing assay) at the selected position (middle position of codon W392X) from samples at the indicated time points in a single experiment.

FIG. 4 is a chart showing editing inhibition with AON IDUA31, having no 2′-OMe modified nucleotides opposite the target adenosine. Editing assays on the duplex RNA were performed in the presence of the indicated concentrations of AON IDUA31. Results are shown as the percentage of edited nucleotide (i.e. nucleotides read as G in the pyrosequencing assay) at the selected position (last position of codon A393) from samples at the indicated time points in a single experiment.

FIG. 5 is a chart showing editing inhibition with AON IDUA32, having 2′-OMe modified nucleotides opposite the target adenosine. Editing assays on the duplex RNA were performed in the presence of the indicated concentrations of AON IDUA32. Results are shown as the percentage of edited nucleotide (i.e. nucleotides read as G in the pyrosequencing assay) at the selected position (last position of codon A393) from samples at the indicated time points in a single experiment.

FIG. 6 shows the sequence of two naturally edited targets (XIAP and 5-HT2RC), with their respective DNA sequences of the ESS regions from 5′ to 3′ (XIAP: SEQ ID NO: 15; HT2RC: SEQ ID NO: 16). Below each of these two sequences are given the sequences of the two varieties of AONs that were tested, from 3′ to 5′ (for XIAP a 24 nt AON with SEQ ID NO: 17 and a 19 nt AON with SEQ ID NO: 18; and for HT2RC a 24 nt AON with SEQ ID NO: 19 and a 19 nt AON with SEQ ID NO: 20). The modifications of each of these AONs are provided in the examples. The edited adenosines are underlined.

FIG. 7 shows the sequencing results of the XIAP ESS in HEK-293 cells after transfection with a variety of AONs competing with the natural ESCS for this target. (A) are the results with the controls showing the natural editing taking place at the two positions indicated by an ‘R’, which represents a mix of an adenosine ‘A’ and a guanine ‘G’ signal. (B) are the results after transfection in HEK-293 cells with four different AONs (here referred to as nEON1, -2, -3, and -4) showing the decrease in the guanine ‘G’ signal at both positions (here given by an ‘A’ at both positions). Only the region surrounding and including the target adenosines is shown: 5′-ARTTGCACART-3′ (SEQ ID NO: 21) in (A) and 5′-AATTGCACAAT-3′ (SEQ ID NO: 22) in (B) above the sequence signals. In all samples from the nEON transfections the sequence software indicated a preference for ‘A’ instead of ‘R’, showing that the guanine signal was lowered significantly. NT = not transfected, Mock = transfection agents only, scrambled = transfection with an unrelated AON.

FIG. 8 shows the sequencing results of the 5-HR2TC ESS in human neuroblastoma SH-SY5Y cells after transfection with a variety of AONs competing with the natural ESCS for this target. (A) are the results with the controls showing the natural editing taking place at the position indicated by an ‘R’, which represents a mix of an adenosine ‘A’ and a guanine ‘G’ signal. (B) are the results after transfection in SH-SY5Y cells with six different AONs (here referred to as nEON1, -2, -3, -4, -5, and -6) showing the decrease in the guanine ‘G’ signal at that position (here given by an ‘A’). Only the region surrounding and including the target adenosine is shown: 5′-GCARTACGTA-3′ (SEQ ID NO: 23) in (A) and (B) above the sequence signals. The sequence software did not give a preference for ‘A’ in the sequence after nEON1 and nEON4 transfection, whereas the sequence software did give a preference for ‘A’ after transfection with nEON2, nEON3, nEON5, and nEON6. NT = not transfected, Mock = transfection agents only, scrambled = transfection with an unrelated AON.

### DETAILED DESCRIPTION

The AONs of the present invention are capable of inhibiting ADAR-mediated deamination of a target adenosine. As discussed, this occurs through a two-step process. In the first step, the AON competes with the ESCS for hybridization with the ESS of the target RNA, to form an AON-ESS duplex. In the second step, the AON-ESS duplex inhibits the ADAR from deaminating the target adenosine. This inhibition can occur through the AON-ESS duplex not being capable of binding to the ADAR or, if the AON-ESS duplex does bind to the ADAR the ADAR catalytic site is prevented from carrying out the catalytic deamination of the target adenosine to inosine. Examples of sequences configured to compete with the ESCS and inhibit ADAR-mediated deamination of the target adenosine are set out below.

To assist with identification of positions, where applicable the following numbering is used. The nucleotide on the AON opposite the target adenosine is nucleotide position 0 and the linkage 5′ from nucleotide position 0 is linkage position 0, and the nucleotide positions and the linkage positions in the AON are both positively (+) and negatively (-) incremented towards the 5′ and 3′ ends respectively. In the case of two or more target adenosines, the numbering can be applied independently to each adenosine, to create overlapping numbering systems.

In one embodiment, the chemical modification comprises a 2′-O ribosyl modification. Different chemical modifications of the nucleotides in the AON (including 2′-O ribosyl modifications) are discussed in great detail in WO2016/097212, WO2017/220751, WO2018/041973, WO2018/134301, WO2019/158475, and WO2019/219581, which are herein incorporated in their entirety.

In one embodiment, the chemical modification comprises a 2′-O ribose modification. In this embodiment, it is preferred that one or more or all of positions +14, +13, +12, +6, +2, +1, 0, -1, -2, -3, -4, and/or -5 comprise a 2′-O ribose modification. In a particularly preferred embodiment, one or more or all of positions +14, +13, +12, 0, -1, -2 and/or -3 comprise a 2′-O ribose modification. In a yet more preferred embodiment, positions 0 and/or -1 comprise a 2′-O ribose modification. In one embodiment, all positions comprise a 2′-O ribose modification. The ribose sugar may be modified by substitution of the 2′-O moietywith a lower alkyl (C1-4, such as 2′-OMe), alkenyl (C2-4), alkynyl (C2-4), methoxyethyl (2′-MOE), or other substituent. Preferred substituents of the 2′ OH group are a methyl, methoxyethyl, F, constrained ethyl (cEt) or 3,3′-dimethylallyl group. The latter is known for its property to inhibit nuclease sensitivity due to its bulkiness, while improving efficiency of hybridization (Angus & Sproat FEBS 1993 Vol. 325, no. 1, 2, 123-7). Alternatively, locked nucleic acid sequences (LNAs), comprising a 2′-4′ intramolecular bridge (usually a methylene bridge between the 2′ oxygen and 4′ carbon) linkage inside the ribose ring, may be applied. Purine nucleobases and/or pyrimidine nucleobases may be modified to alter their properties, for example by amination or deamination of the heterocyclic rings. The exact chemistries and formats may depend from oligonucleotide construct to oligonucleotide construct and from application to application, and may be worked out in accordance with the wishes and preferences of those of skill in the art. In a preferred embodiment, the 2′-O ribose modification is a 2′-OMe and/or 2′-MOE ribose modification. The modifications can be exclusively 2′-OMe or exclusively 2′-MOE, or can be a mixture of 2′-OMe and 2′-MOE, or can be a mixture of 2′-OMe and/or 2′-MOE and/or unmodified 2′-O and/or further 2′-O modifications. It is particularly preferred that the position opposite the target adenosine comprises a 2′-OMe or 2′-MOE modification, preferably a 2′-MOE modification.

In one embodiment, the chemical modification comprises an unlocked nucleic acid (UNA). In this modification, there is no carbon-carbon bond between the ribose 2′ and 3′ carbon atoms. UNA ribose modifications therefore increase the local flexibility in oligonucleotides. In addition to inhibiting ADAR-mediated deamination, UNAs can lead to effects such as improved pharmacokinetic properties through improved resistance to degradation. UNAs can also decrease toxicity. In this embodiment, it is preferred that an unlocked nucleic acid modification is present at one or more or all of nucleotide positions +14, +13, +12, +2, 0, and -3. The UNA modification may also exist in addition to modifications to the ribose 2′ group, either at positions different to the UNA modifications or at the same positions as the UNA modifications. The ribose 2′ groups in the AON can be independently selected from 2′-H (i.e. DNA), 2′-OH (i.e. RNA), 2′-OMe, 2′-MOE, 2′-F, or 2′-4′-linked (i.e. a locked nucleic acid or LNA). The 2′-4′ linkage can be selected from linkers known in the art, such as a methylene linker or constrained ethyl linker.

In one embodiment one or more or all internucleoside linkages are modified. That is, they can be a phosphodiester wherein the OH group of the phosphodiester has been replaced by alkyl, alkoxy, aryl, alkylthio, acyl, -NR1R1, alkenyloxy, alkynyloxy, alkenylthio, alkynylthio, -S-Z+, -Se-Z+, or- BH3-Z+, and wherein R1 is independently hydrogen, alkyl, alkenyl, alkynyl, or aryl, and wherein Z+ is ammonium ion, alkylammonium ion, heteroaromatic iminium ion, or heterocyclic iminium ion, any of which is primary, secondary, tertiary or quaternary, or Z is a monovalent metal ion, and is preferably a phosphorothioate (PS) linkage. In a preferred embodiment the internucleotide linkage modification is at position +8, +7, +6, +5, -4 and/or -5.

Where a PS linkage modification is at position 10, it is preferably in the R configuration.

Where a PS linkage modification is at position +4, -1, -3 and/or -6, it is preferably in the S configuration. The preference for specific PS stereochemistry at different positions is described further in WO2019/219581.

In one embodiment, one or more or all internucleoside linkages are modified to be a methylphosphonate (MP) linkage. It is preferred that the AON comprises one or more MP linkages at linkage positions 0, -1, -2, -3, -4, -5 and/or -6, more preferably the AON comprises MP linkages at linkage positions -0 and/or -2. In one embodiment, the AON comprises a single MP linkage. Especially preferred is the aspect that the MP linkage renders the AON more stable than an AON lacking that MP linkage when compared in an in vitro stability assay. The preference for specific MP linkages is described in PCT/EP2020/059369 (unpublished).

In one embodiment, the chemical modification comprises a phosphonoacetate linkage modification. Phosphonoacetate linkages are well-referenced and highly relevant RNA modifications for therapeutically optimized oligonucleotides. In addition to inhibiting ADAR-mediated deamination, an important role of this modification is to protect the polymer from nuclease-mediated degradation. Phosphonoacetate modifications can also be responsible for improved uptake of the EONs into target cells. In this embodiment, it is preferred that a phosphonoacetate modification is present at one or more or all of linkage positions +13, +12, +11, +8, +7, +6, -1, -2, -3, -4 and -5, particularly at one or more or all of linkage positions +13, +12, +7, -4 and -5. As discussed in PCT/EP2020/053283 (unpublished), phosphonoacetate modifications at these positions can cause disruption of binding with ADAR. The internucleotide linkages that are not phosphonoacetate internucleotide linkages are preferably an unmodified phosphodiester or another linkage such as PS, phosphodithioate, 3′-methylenephosphonate, 5′-methylenephosphonate, 3′-phosphoroamidate and the like.

The AON can comprise one or more mismatches, wobbles and/or bulges with the ESS, as long as the AON still competes with the ESCS for hybridisation of the ESS. In a preferred embodiment, the AON comprises no mismatches, wobbles and/or bulges with the ESS, and in particular comprises no mismatches, wobbles and/or bulges with the ESS at the target adenosine.

The AON can comprise further sequence in addition to the sequence that hybridises with the ESS, as long as the AON still competes with the ESCS for hybridisation of the ESS.

When the nucleotide opposite the target adenosine is a uridine or a deoxyuridine, the EON may be 100% complementary and not have any mismatches, wobbles or bulges in relation to the target RNA. The AON of the invention will usually comprise the normal nucleotides A, G, U and C, but may also include inosine (I), for example instead of one or more G nucleotides.

In one particular embodiment of the present invention, the AON is at least 10, 11, 12, 13, 14, 15, 16, 17, 18, 19, 20, 21, 22, 23, 24, 25, 26, 27, 28, 29, 30, 31, or 32 nucleotides in length. Preferably, the AON is shorter than 100 nucleotides, more preferably shorter than 60 nucleotides, and even more preferably, the AON comprises 18 to 70 nucleotides, 18 to 60 nucleotides, or 18 to 50 nucleotides.

Preferably the AON of the present invention does not have a portion that allows the AON in itself to fold into an intramolecular hairpin or other type of (stem) loop structure (herein also referred to as “auto-looping” or “self-looping”), and which may potentially act as a structure that sequesters ADAR.

The invention concerns the modification of target RNA sequences in eukaryotic, preferably metazoan, more preferably mammalian cells. In principle the invention can be used with cells from any mammalian species, but it is preferably used with a human cell. The invention can be used with cells from any organ e.g. skin, lung, heart, kidney, liver, pancreas, gut, muscle, gland, eye, brain, blood and the like. The cell can be located in vitro or in vivo. One advantage of the invention is that it can be used with cells in situ in a living organism, but it can also be used with cells in culture. In some embodiments, cells are treated ex vivo and are then introduced into a living organism (e.g. re-introduced into an organism from whom they were originally derived).

The amount of oligonucleotide to be administered, the dosage and the dosing regimen can vary from cell type to cell type, the disease to be treated, the target population, the mode of administration (e.g. systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change.

One suitable trial technique involves delivering the oligonucleotide construct to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell’s target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively the change may be assessed on the level of the protein (length, glycosylation, function or the like), or by some functional read-out, such as a(n) (inducible) current, when the protein encoded by the target RNA sequence is an ion channel, for example. In the case of CFTR function, an Ussing chamber assay or an NPD test in a mammal, including humans, are well known to a person skilled in the art to assess restoration or gain of function.

Oligonucleotides of the invention are particularly suitable for therapeutic use, and so the invention provides a pharmaceutical composition comprising an oligonucleotide of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g. syringe, inhaler, nebuliser) which includes a pharmaceutical composition of the invention. Especially when the AON is to be delivered to the epithelial cells of the airways (for instance to prevent or treat a respiratory viral infection, e.g. in the case of an influenza- or coronavirus) the use of a nebuliser to administer the AON to the subject in need thereof, is preferred.

The invention also relates to a method for inhibiting the deamination of at least one specific target adenosine present in a target RNA sequence in a cell, the method comprising the steps of: providing the cell with an AON according to the invention; allowing uptake by the cell of the AON; and allowing annealing of the AON to the target RNA sequence; and optionally identifying the presence of the adenosine or inosine in the RNA sequence.

Introduction of the AON according to the present invention into the cell is performed by general methods known to the person skilled in the art. The read-out of the effect (e.g. alteration or prevention of alteration of the target RNA sequence) can be monitored through different ways. Hence, the identification step of whether deamination of the target adenosine has taken place depends generally on the position of the target adenosine in the target RNA sequence, and the effect that is incurred by the presence of the adenosine (point mutation, early stop codon). Hence, in a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by deamination; or using a functional read-out, wherein the target RNA before deamination encodes a functional, full length, elongated and/or wild type protein. The identification of prevention of deamination into inosine may also be a functional read-out, for instance an assessment on whether a functional protein is present, or even the assessment that a disease that is caused by the deamination is (partly) reversed. The functional assessment for each of the diseases mentioned herein will generally be according to methods known to the skilled person. A very suitable manner to identify the presence of adenosine or an inosine after deamination of the target adenosine is of course RT-PCR and sequencing, using methods that are well-known to the person skilled in the art.

The AON according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 µg/kg to about 100 mg/kg, preferably from about 10 µg/kg to about 10 mg/kg, more preferably from about 100 µg/kg to about 1 mg/kg. Administration may be by inhalation (e.g. through nebulization), intranasally, orally, by injection or infusion, intravenously, subcutaneously, intra-dermally, intra-cranially, intravitreally, intramuscularly, intra-tracheally, intra-peritoneally, intra-rectally, and the like. Administration may be in solid form, in the form of a powder, a pill, or in any other form compatible with pharmaceutical use in humans. The invention is particularly suitable for treating genetic diseases, such as cystic fibrosis. The invention is also particularly suitable for treating viral infections, such as respiratory viral infections, more preferably, viral infections caused by coronaviruses such as SARS-CoV-1, MERS-CoV, and SARS-CoV-2, and derivatives thereof.

In some embodiments the oligonucleotide construct can be delivered systemically, but it is more typical to deliver an oligonucleotide to cells in which the target sequence’s phenotype is seen.

### Definitions of Terms as Used Herein

The terms ‘adenine’, ‘guanine’, ‘cytosine’, ‘thymine’, ‘uracil’ and ‘hypoxanthine’ (the nucleobase in inosine) as used herein refer to the nucleobases as such.

The terms ‘adenosine’, ‘guanosine’, ‘cytidine’, ‘thymidine’, ‘uridine’ and ‘inosine’ refer to the nucleobases linked to the (deoxy)ribosyl sugar.

The term ‘nucleoside’ refers to the nucleobase linked to the (deoxy)ribosyl sugar.

The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), an unlocked nucleic acid, a nucleotide including a linker comprising a phosphodiester, phosphonoacetate, phosphotriester, phosphoro(di)thioate, methylphosphonates, phosphoramidate linkers, and the like.

Sometimes the terms adenosine and adenine, guanosine and guanine, cytidine and cytosine, uracil and uridine, thymine and thymidine/uridine, inosine and hypo-xanthine, are used interchangeably to refer to the corresponding nucleobase on the one hand, and the nucleoside or nucleotide on the other.

Sometimes the terms nucleobase, nucleoside and nucleotide are used interchangeably, unless the context clearly requires differently. The terms ‘ribonucleoside’ and ‘deoxyribonucleoside’, or′ribose’ and ‘deoxyribose’ are as used in the art.

Whenever reference is made to an ‘oligonucleotide’, both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I. In a preferred aspect, the EON of the present invention is an oligoribonucleotide that may comprise chemical modifications and may include deoxynucleotides (DNA) at certain specified positions. Terms such as oligonucleotide, oligo, ON, oligonucleotide composition, antisense oligonucleotide, AON, and RNA (antisense) oligonucleotide may be used herein interchangeably.

Whenever reference is made to nucleotides in the oligonucleotide construct, such as cytosine, 5-methylcytosine, 5-hydroxymethylcytosine and β-D-Glucosyl-5-hydroxymethylcytosine are included; when reference is made to adenine, N6-Methyladenine and 7-methyladenine are included; when reference is made to uracil, dihydrouracil, 4-thiouracil and 5-hydroxymethyluracil are included; when reference is made to guanine, 1-methylguanine is included.

Whenever reference is made to nucleosides or nucleotides, ribofuranose derivatives, such as 2′-desoxy, 2′-hydroxy, and 2′-O -substituted variants, such as 2′-O-methyl, are included, as well as other modifications, including 2′-4′ bridged variants.

Whenever reference is made to oligonucleotides, linkages between two mono-nucleotides may be phosphodiester linkages as well as modifications thereof, including, phosphonoacetate, phosphodiester, phosphotriester, phosphoro(di)thioate, methylphosphonate, phosphor-amidate linkers, and the like.

The term ‘comprising’ encompasses ‘including’ as well as ‘consisting’, e.g. a composition comprising X′ may consist exclusively of X or may include something additional, e.g. X + Y.

The term ‘about’ in relation to a numerical value x is optional and means, e.g. x±10%.

The word ‘substantially’ does not exclude ‘completely’, e.g. a composition which is ‘substantially free from Y’ may be completely free from Y. Where relevant, the word ‘substantially’ may be omitted from the definition of the invention.

The term “complementary” as used herein refers to the fact that the AON hybridizes under physiological conditions to the target sequence. The term does not mean that each nucleotide in the AON has a perfect pairing with its opposite nucleotide in the target sequence. In other words, while an AON may be complementary to a target sequence, there may be mismatches, wobbles and/or bulges between AON and the target sequence, while under physiological conditions that AON still hybridizes to the target sequence competitively with the ESCS. The term “substantially complementary” therefore also means that despite the presence of the mismatches, wobbles, and/or bulges, the AON has enough matching nucleotides between AON and target sequence that under physiological conditions the AON hybridizes to the target RNA. As shown herein, an AON may be complementary, but may also comprise one or more mismatches, wobbles and/or bulges with the target sequence, if under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that the majority of stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. Mismatched nucleotides are G-A, C-A, U-C, A-A, G-G, C-C, U-U pairs. In some embodiments EONs of the present invention comprise fewer than four mismatches, for example 0, 1 or 2 mismatches. Wobble base pairs are: G-U, I-U, I-A, and I-C base pairs.

### EXAMPLES

### Example 1: Chemically Modified AONs Do Not Allow Editing of a Target Adenosine When Duplexed With an ESS

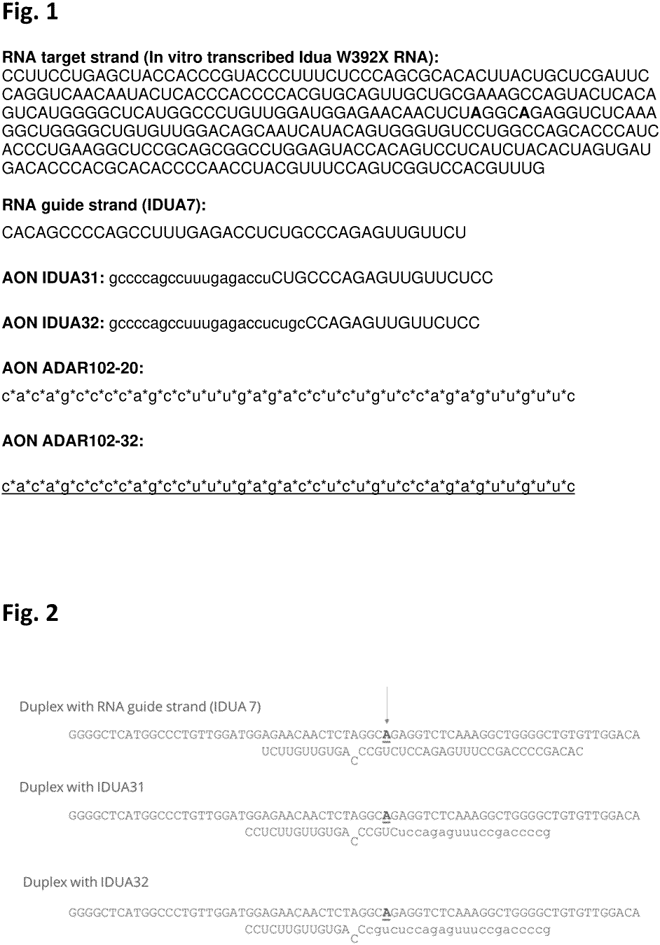

To investigate whether chemical modifications are indeed an effective method to prevent editing, when duplexed with the ESS of a target RNA, the inventors tested this in an in vitro editing assay. As a positive control, an RNA duplex mimicking a natural ADAR editing target was generated by annealing an in vitro transcribed RNA sequence (the edited, or ESS, strand) from the mouse Idua (W392X) mRNA (as discussed in more detail in, for example, WO2017220751 or WO2018041973) to a partially complementary RNA oligonucleotide (the guide strand, or ESCS; IDUA7; see FIGS. 1 and 2). The test subjects were two antisense oligonucleotides (AONs) that were fully modified with 2′-OMe and PS (ADAR102-20) or with 2′-MOE and PS (ADAR102-32), and in order to test their inherent ability to inhibit editing, they were similarly duplexed directly with the ESS of the target RNA.

The RNA guide strand (IDUA7), ADAR102-20 and ADAR102-32 were separately annealed to the mIDUA target RNA. Annealing was done in a buffer (5 mM Tris-Cl pH 7.4, 0.5 mM EDTA and 10 mM NaCl) with a concentration of 1 nM target RNA and 3 nM IDUA7, ADAR102-20 or ADAR102-32. The samples were heated at 95° C. for 3 min and then slowly cooled down to room temperature. Next, the editing reaction was carried out by mixing with protease inhibitor (cOmpleteTM, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 3 mM MgCl₂ and 0.5 mM DTT). The reaction was started by adding purified human ADAR2, which was produced by GenScript, to a final concentration of 3 nM into the mix and incubated for predetermined time points (0, 4, 10, or 25 min) at 37° C. The reaction was stopped by adding 190 µL boiling water and then the mixture was incubated for 5 min at 95° C. Then, 6 µl of the stopped reaction mixture was used as template for cDNA synthesis using Maxima reverse transcriptase kit with random hexamer primers (Thermo Fisher) according to the manufacturer’s instructions in a total volume of 20 µl and using an extension temperature of 62° C.

Products were amplified for pyrosequencing analysis by PCR, using the Amplitaq gold 360 DNA Polymerase kit (Applied Biosystems) according to the manufacturer’s instructions, with 1 µl of the cDNA as template. The following primers were used at a concentration of 10 µM: Pyroseq Fwd2 IDUA, 5′-AGTACTCACAGTCATGGGGCTCA-3′ (SEQ ID NO: 8), and Pyroseq Rev2 IDUA Biotin, 5′-GCCAGGACACCCACTGTATGAT-3′ (SEQ ID NO: 9). The latter primer also contains a biotin conjugated to its 5′ end, as required for the automatic processing during the pyrosequencing reactions. The PCR was performed using the following thermal cycling protocol: Initial denaturation at 95° C. for 5 min, followed by 45 cycles of 95° C. for 30 sec, 60° C. for 30 sec and 72° C. for 30 sec, and a final extension of 72° C. for 7 min.

As inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and the following data analysis were performed by the PyroMark Q48 Autoprep instrument (QIAGEN) following the manufacturer’s instructions, with 10 µl input of the PCR product and 4 µM of the following sequencing primer: IDUA-Seq2, 5′-TGGGGCTCATGGCCCT -3′ (SEQ ID NO: 10). The settings specifically defined for this target RNA strand included two sets of sequence information. The first of these defines the sequence for the instrument to analyse, in which the potential for a particular position to contain either an adenosine or a guanosine is indicated by a “/”: GTTGGATGGAGAACAACTCTA/GGGCAGAGGTCTCAA/GAGGCTGGGGCTGTGTTGGACAG (SEQ ID NO: 11). The second set defines the order in which the sequencing reagents corresponding to each nucleotide are to be dispensed, and also includes blank controls (i.e. nucleotides that should not be incorporated at that particular position), which is used by the instrument to define the background signal. The dispensation order was defined for this analysis as follows, with the blank controls highlighted:

CGTGATGAGACACTCGTAGCAGAGTCTGCAGAGCTGCA (SEQ ID NO
: 13).

The analysis performed by the instrument provides the results for the selected nucleotide as a percentage of adenosine and guanosine detected in that position, and the extent of A-to-I editing at a chosen position (here, specifically the A in codon W392X) will therefore be measured by the percentage of guanosine in that position.

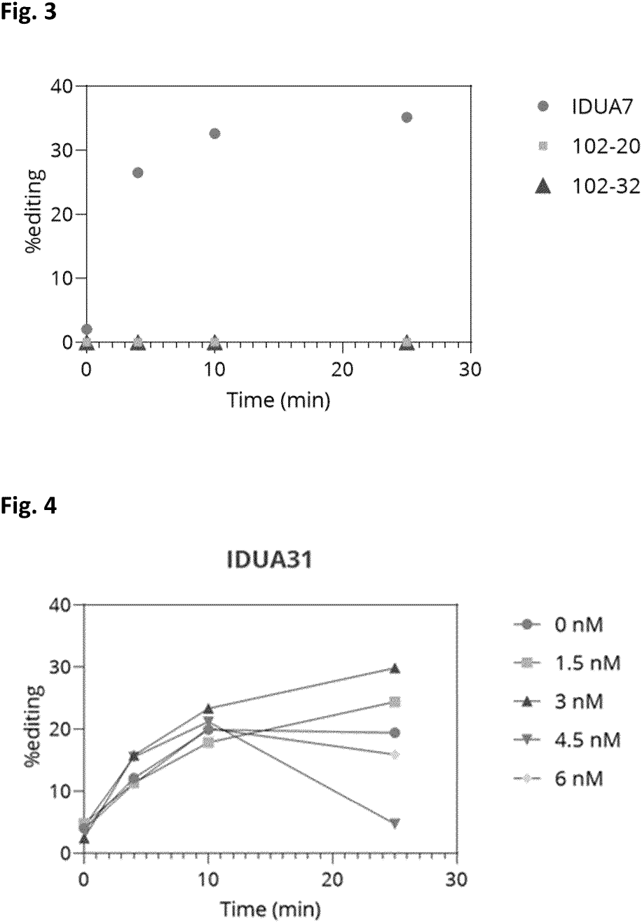

The results shown in FIG. 3 clearly indicate that while the target A was efficiently edited in the RNA duplex using IDUA7, no editing was observed when the chemically modified AONs 102-20 and 102-32 were annealed directly to the ESS, indicating the chemical modifications are efficient at inhibiting ADAR2 binding and/or catalytic activity.

### Example 2: Inhibition of A-to-I Editing by a Chemically Modified AON That Prevents the Formation of an Editing Capable Duplex by Competing With an Editing-Site Complementary Strand

To investigate whether an AON can compete with an ESCS and inhibit ADAR-mediated editing, and whether chemical modifications are necessary for this inhibition, an RNA duplex mimicking a natural ADAR editing target was generated by annealing an in vitro transcribed RNA sequence (the edited, or ESS, strand) from the mouse Idua (W392X) mRNA to a partially complementary RNA oligonucleotide (the guide strand, or ESCS; IDUA7; see FIGS. 1 and 2). An adenosine located in the wobble position of the alanine codon, A393, of the mouse Idua mRNA was chosen as the test subject for inhibition, and two AONs with a different extent of 2′-OMe modifications were selected such that one of them (IDUA31) had a region of unmodified RNA nucleotides that would anneal to the sequence including the adenosine in codon A393, while in the other (IDUA32) the nucleotides in the immediate vicinity of the editing site were changed to 2′-OMe-modified nucleotides (FIGS. 1 and 2). The inhibitory effect of these AONs were compared in an RNA editing assay as follows. First, the RNA guide strand was annealed to the mIDUA target RNA either in the absence of presence of varying amounts of the AONs. Annealing was done in a buffer (5 mM Tris-Cl pH 7.4, 0.5 mM EDTA and 10 mM NaCl) with a concentration of 1 nM target RNA and 3 nM RNA guide strand, and either 0, 1.5, 3, 4.5 or 6 nM AON. The samples were heated at 95° C. for 3 min and then slowly cooled down to room temperature. Next, the editing reaction was carried out. The annealed EON and target RNA were mixed with protease inhibitor (cOmpleteTM, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 3 mM MgCl₂ and 0.5 mM DTT). The reaction was started by adding purified human ADAR2, which was produced by GenScript, to a final concentration of 3 nM into the mix and incubated for predetermined time points (0, 4, 10, or 25 min) at 37° C. The reaction was stopped by adding 190 µL boiling water and then the mixture was incubated for 5 min at 95° C. Then, 6 µl of the stopped reaction mixture was used as template for cDNA synthesis using Maxima reverse transcriptase kit with random hexamer primers (Thermo Fisher) according to the manufacturer’s instructions in a total volume of 20 µl and using an extension temperature of 62° C.

Products were amplified for pyrosequencing analysis by PCR, using the Amplitaq gold 360 DNA Polymerase kit (Applied Biosystems) according to the manufacturer’s instructions, with 1 µl of the cDNA as template. The following primers were used at a concentration of 10 µM: Pyroseq Fwd2 IDUA and Pyroseq Rev2 IDUA Biotin (see example 1). The PCR was performed as described in example 1.

As inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and the following data analysis were performed by the PyroMark Q48 Autoprep instrument (QIAGEN) following the manufacturer’s instructions, with 10 µl input of the PCR product and 4 µM of sequencing primer IDUA-Seq2. The settings specifically defined for this target RNA strand included two sets of sequence information. The first of these defines the sequence for the instrument to analyse, in which the potential for a particular position to contain either an adenosine or a guanosine is indicated by a “/”: GTTGGA/GTGGA/GGAACAA/GCTCTA/GGGCA/GGAGGTCTCAAA/GGGCTGGGGCTGTGTTGGA/G CAG (SEQ ID NO: 14). The second set defines the order in which the sequencing reagents corresponding to each nucleotide are to be dispensed, and also includes blank controls (i.e. nucleotides that should not be incorporated at that particular position), which is used by the instrument to define the background signal. The dispensation order was defined for this analysis as follows, with the blank controls highlighted: AGCTGACTGGAGAGCGAGCTCATAGTCAGAGTCTGCGAGAGCTGGCTGTGCTGACA (SEQ ID NO: 12). The analysis performed by the instrument provides the results for the selected nucleotide as a percentage of adenosine and guanosine detected in that position, and the extent of A-to-I editing at a chosen position will therefore be measured by the percentage of guanosine in that position.

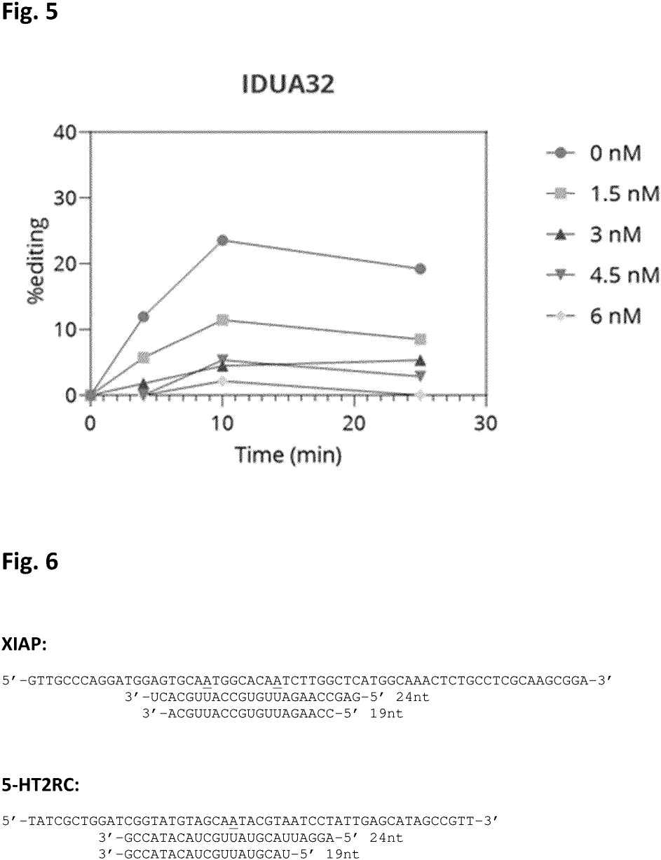

The pyrosequencing results (percentage of guanosine) for the selected target adenosine are plotted in FIG. 4 and clearly show that, when using an AON with RNA nucleotides that can anneal to the A393 codon (IDUA31), the editing of the adenosine in codon A393 remains essentially on the same level regardless of the concentration of the AON used. It is noted that the last time point with 4.5 nM is likely to be the result of a technical error in preparation of this sample for measurement. In contrast, an AON with 2′-OMe nucleotides in this region (IDUA32) clearly inhibits editing of the adenosine in A393 in a concentration-dependent manner (FIG. 5), with 6 nM AON resulting in almost complete inhibition of editing. Thus, the AON can compete with the RNA guide strand (ESCS) for binding to the edited strand (ESS) and inhibit editing, and this inhibition depends on the chemical modifications within the AON. Furthermore, the data suggests that a chosen site can either be edited or not edited depending on the chemical modifications within the AON, which allows the AON to be designed in a way to specifically inhibit editing of only some of the adenosines in the target region after competing away the original guide strand.

### Example 3: Inhibition of A-to-I Editing by a Chemically Modified AON in Cells

To further investigate whether the inhibition of RNA could be achieved in a setting with endogenous ADAR, the inventors selected two targets that were known to be edited in nature: XIAP and 5-HT2RC, and to see whether transfection with chemically modified AONs targeting their respective ESS sequences would lower the level of editing seen under normal circumstances. XIAP was primarily chosen because of the location of the editing sites in the 3′ UTR. 5-HT2RC is a potential therapeutic target (Dracheva et al. 2008).

For XIAP, HEK-293 cells were plated in a 6-well plate 24 h prior to transfections in a density of 2.5×10⁵ cells per well and cultured in Dulbecco’s Modified Eagle Medium (Gibco) supplemented with 10% FBS. Cells were either not transfected (NT), treated with transfection reagent only (Mock), transfected with an unrelated non-targeting oligo (scrambled control; 100 nM of a 24-mer oligonucleotide) or transfected with final concentration of 100 nM of the following four XIAP specific AONs (see FIG. 6 for the complementarity to the target sequence):

nEON1 mer):

mG*mA*mG*mC*mC*mA*mA*mG*mA*mU*mU*mG*mU*mG*mC*mC*mA
*mU*mU*mG* mC*mA*mC*mU

nEON2 mer):

moeG*moeA*moeG*moeC*moeC*moeA*moeA*moeG*moeA*moeT*
moeT*moeG*moeT*moeG*moeC*moeC*moeA*moeT*moeT*moeG*
moeC*moeA*moeC*moeT

nEON3 mer):

mG*mA*InG*mC*mC*InA*mA*mG*InA*mU*mU*InG*mU*mG*InC*
mC*mA*InT*mU*mG*InC*mA*mC*mU

nEON4 mer):

mC*mC*mA*mA*mG*mA*mU*mU*mG*mU*mG*mC*mC*mA*mU*mU*mG
*mC*mA

The AONs above are given from 5′ to 3′. The asterisk (*) indicates a phosphorothioate linkage. ‘m’ indicates a 2′-OMe modification of the sugar moiety. ‘moe’ indicates a 2′-MOE modification of the sugar moiety. ‘In’ indicates a locked nucleic acid (LNA) modification. Since all nucleosides are modified at the 2′ position or are LNA, there is no distinction between RNA or DNA. The reason for the U or T notation is that the 2′-OMe modified AONs are built with 2′-OMe/U building blocks and the 2′-MOE modified AONs were built with 2′-MOE/T building blocks (2′-OMe/T building blocks were not applied in the manufacturing).

Transfections were performed using Lipofectamine 2000 (Invitrogen) following the manufacturer’s protocol at a ratio of 2 µl Lipofectamine 2000 to 1 µg EON). Prior to transfection, medium was replaced to 1.7 mL/well fresh medium and a transfection mix (300 µl) was added, making a total of 2 mL/well. After 24 h medium was removed and cells were washed once with 1X PBS and then 350 µl of Trizol was added to each well for cell lysis. The lyzed cells were then collected and RNA was extracted using the Direct-zol RNA MiniPrep (Zymo Research) kit according to the manufacturer’s instructions. RNA concentrations were measured using the Nanodrop and 500 ng RNA was used for cDNA synthesis. cDNA synthesis was performed using the Maxima reverse transcriptase kit (Thermo Fisher) according to the manufacturer’s instructions, with a combination of random hexamer and oligo-dT primers. Then, cDNA was subjected to RT-PCR using the Amplitaq Gold 360 DNA Polymerase 1000U kit (Applied Biosystems) according to the manufacturer’s instructions to amplify the target region. The following primers were used:
XIAP PCR forward: 5′-CTACTCCGGAGCCTGAGGT-3′ (SEQ ID NO: 24)
XIAP PCR reverse: 5′-TGGGAGATGAATCACTTGAGGT-3′ (SEQ ID NO: 25)
XIAP sequencing: 5′-CAGAAATTCAAGACTAGCCTGGC-3′ (SEQ ID NO: 26)

For 5-HT2RC, human neuroblastoma SH-SY5Y cells were plated in a 6-well plate 24 h prior to transfections in a density of 3×10⁵ cells per well and were cultured in Eagle’s Minimum Essential Medium F12 medium (Gibco) supplemented with 15% FBS and 1% non-essential amino acids. Cells were either not transfected (NT), treated with transfection reagent only (Mock), transfected with an unrelated non-targeting oligo (scrambled control; 100 nM of a 24-mer oligonucleotide) or transfected with final concentration of 100 nM of the following four AONs (see FIG. 6 for the complementarity to the target sequence):

nEON1 mer):

mA*mG*mG*mA*mU*mU*mA*mC*mG*mU*mA*mU*mU*mG*mC*mU*mA
*mC*mA*mU*mA*mC*mC*mG

nEON2 mer):

moeA*moeG*moeG*moeA*moeT*moeT*moeA*moeC*moeG*moeT*
moeA*moeT*moeT*moeG*moeC*moeT*moeA*moeC*moeA*moeT*
moeA*moeC*moeC*moeG

nEON3 mer):

mA*mG*InG*mA*mU*InT*mA*mC*InG*mU*mA*InT*mU*mG*InC*
mU*mA*InC*mA*mU*InA*mC*mC*mG

nEON4 mer):

mU*mA*mC*mG*mU*mA*mU*mU*mG*mC*mU*mA*mC*mA*mU*mA*mC
*mC*mG

nEON5 mer):

moeT*moeA*moeC*moeG*moeT*moeA*moeT*moeT*moeG*moeC*
moeT*moeA*moeC*moeA*moeT*moeA*moeC*moeC*moeG

nEON6 mer):

mU*mA*InC*mG*mU*InA*mU*mU*InG*mC*mU*InA*mC*mA*InT*
mA*mC*InC*mG

The AONs above are given from 5′ to 3′. The asterisk (*) indicates a phosphorothioate linkage. ‘m’ indicates a 2′-OMe modification of the sugar moiety. ‘moe’ indicates a 2′-MOE modification of the sugar moiety. ‘In’ indicates a locked nucleic acid (LNA) modification.

Since all nucleosides are modified at the 2′ position or are LNA, there is no distinction between RNA or DNA. The reason for the U or T notation is that the 2′-OMe modified AONs are built with 2′-OMe/U building blocks and the 2′-MOE modified AONs were built with 2′-MOE/T building blocks (2′-OMe/T building blocks were not applied in the manufacturing).

Transfections, culturing, RNA extraction, cDNA synthesis and PCR were performed as outlined above. PCR products were analysed by Sanger sequencing in order to detect inhibition of A-I editing. The following primers were used:
5-HT2RC PCR forward: 5′- GGCAATCCTTTATGATTATGTCTGG -3′ (SEQ ID NO: 27)
5-HT2RC PCR reverse: 5′-TGCCCAAACAATAGCAATCTTCA-3′ (SEQ ID NO: 28)
5-HT2RC sequencing: 5′-TCATGATGGCCTTAGTCCGC-3′ (SEQ ID NO: 29)

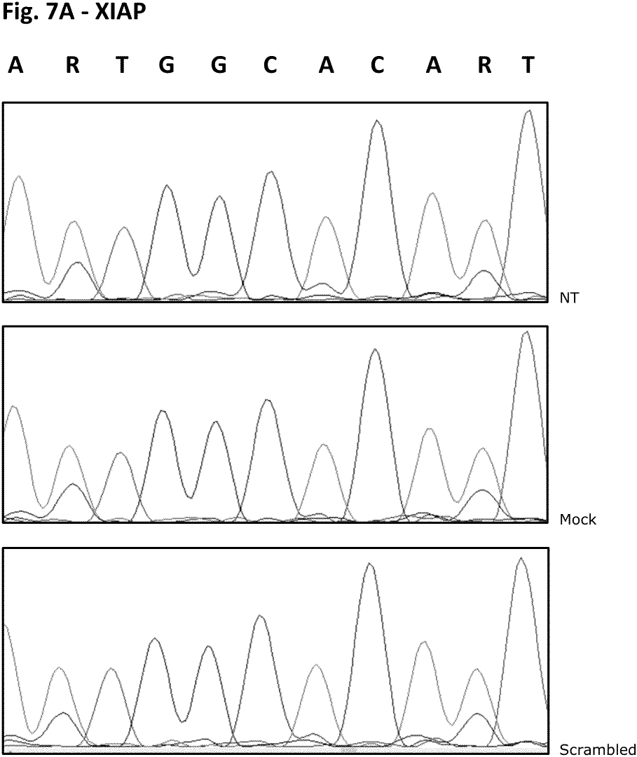

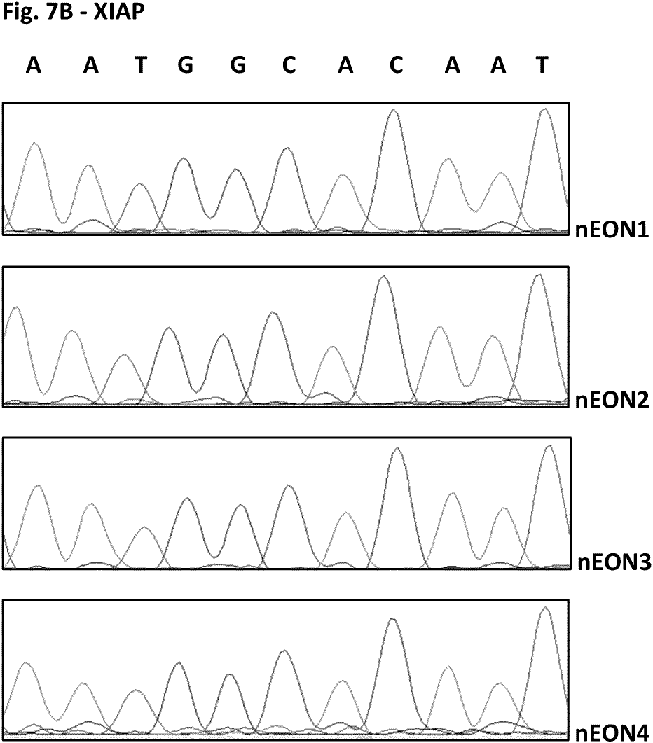

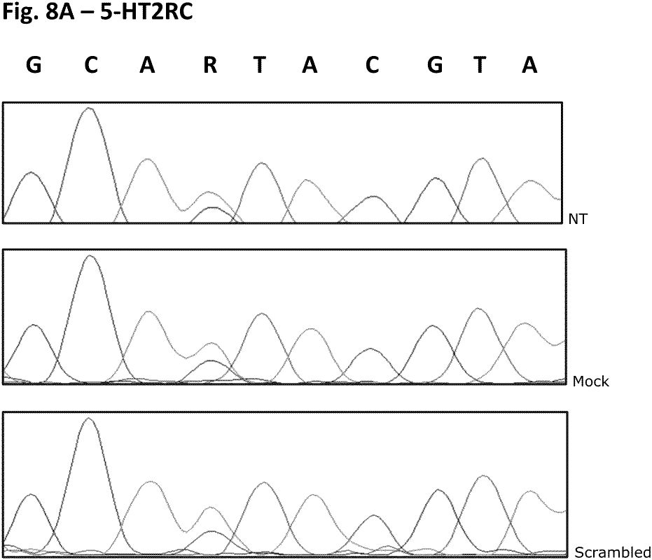

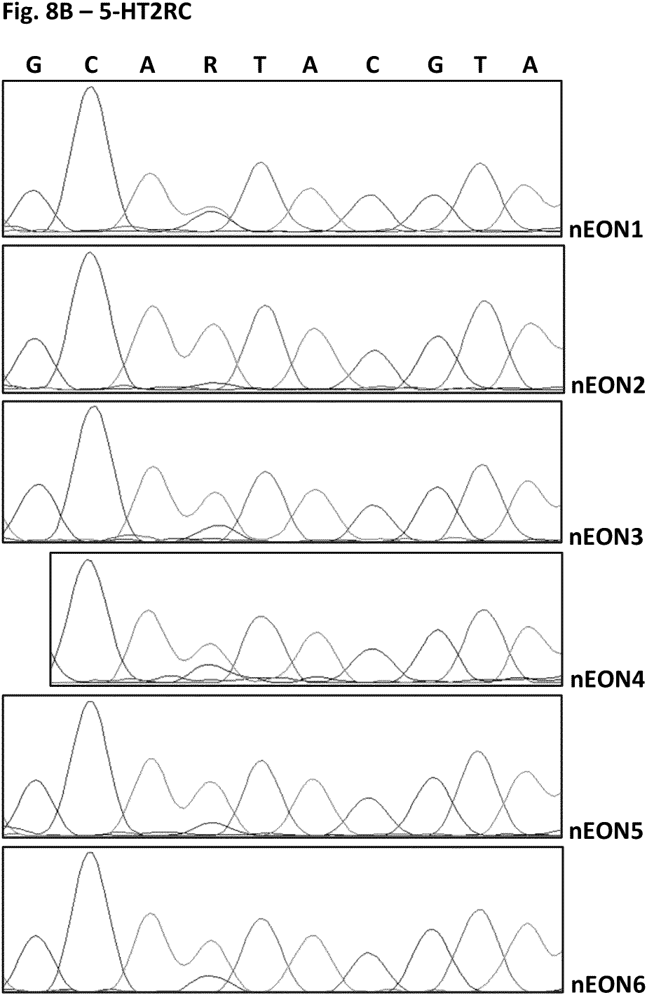

FIG. 7 shows the sequencing results of the XIAP experiment, in which FIG. 7A shows the editing taking place at the two positions indicated with an ‘R’, which shows a mix of an adenosine and a guanine signal at these positions, indicating that in these cells, when not using targeting AONs, RNA editing is taking place in a natural way, with endogenous RNA editing enzymes. FIG. 7B shows the same sequence, but now at the positions of the ‘R’ the indication of an ‘A’, which means that the sequencing software determines an adenosine at these positions rather than a mixed signal. The peak of the guanine seen in the control samples is clearly down. FIG. 8 shows the sequencing results of the 5-HT2RC experiment, in which FIG. 8A shows the editing taking place at the single position indicated with an ‘R’, which shows a mix of an adenosine and a guanine signal at this position, indicating that in these cells, when not using targeting AONs, RNA editing is taking place in a natural way, with endogenous RNA editing enzymes. FIG. 8B shows the same sequence (except in the case of nEON4, that lacks the 5′ guanine), but now at the position of the ‘R’ the indication of an ‘A’, which means that the sequencing software determines an adenosine at these positions rather than a mixed signal. This was seen after transfections with nEN2, -3, -5, and -6, whereas nEON1 and -4 were not significantly decreasing the guanine signal, since the sequencing software still indicated an ‘R’ at the editing site.

These experiments clearly show that the inventors were able to inhibit naturally occurring RNA editing in cells, with endogenous RNA editing enzymes, on a natural target, by targeting chemically modified AONs to the ESS in two instances, with 5-HT2RC showing that inhibition can be achieved within a coding region of a (pre-)mRNA, and with XIAP showing that inhibition is similarly possible in a non-coding region.

1. An antisense oligonucleotide (AON) capable of inhibiting ADAR-mediated deamination of a target adenosine present in an editing-site sequence (ESS) of an endogenous target RNA molecule, wherein under physiological conditions the ESS would hybridize with an editing-site complementary sequence (ESCS) of an endogenous RNA molecule to form a double stranded RNA complex, wherein the AON comprises a sequence configured to compete with the ESCS for hybridization with the ESS.

2. The AON of claim 1, wherein the nucleotide in the AON opposite the target adenosine and/or the nucleotides in the AON opposite the nucleotides surrounding the target adenosine is/are chemically modified to compete with the ESCS and inhibit the ADAR-mediated deamination of the target adenosine.

3. The AON of claim 2, wherein the nucleotides surrounding the target adenosine consist of the adjacent two bases in the 3′ direction and the adjacent two bases in the 5′ direction.

4. The AON of claim 1, wherein the nucleotides in the AON opposite all adenosines in the target RNA molecule are each chemically modified to compete with the ESCS and inhibit the ADAR-mediated deamination of each corresponding adenosine in the target RNA molecule.

5. The AON of claim 2, wherein the chemical modification comprises a 2′-O ribosyl derivative.

6. The AON of claim 5, wherein the 2′-O ribosyl derivative is 2′-O-alkyl, 2′-O-methyl (2′-OMe), 2′-O-methoxyethyl (2′-MOE), a locked nucleic acid, or a constrained nucleic acid (cEt).

7. The AON of claim 1, wherein the nucleotide opposite the target adenosine is uridine.

8. The AON of claim 1, wherein the ADAR is ADAR1 or ADAR2.

9. The AON claim 1, wherein the ESS and ESCS are located on the same RNA molecule.

10. The AON of claim 1, wherein under physiological conditions the ESS would hybridize with the ESCS to form a double stranded RNA complex in a cell.

11. The AON of claim 1, wherein the ESS is of a pre-messenger RNA, a messenger RNA, a long non-coding RNA, a ribosomal RNA, a transfer RNA, a long non-coding RNA or a miRNA or a precursor of any of the foregoing RNAs.

12. The AON of claim 1, wherein the target adenosine is located within a coding sequence of an RNA molecule.

13. A pharmaceutical composition comprising AON of claim 1, and a pharmaceutically acceptable carrier, excipient and/or adjuvant.

14. A method of inhibiting ADAR-mediated deamination of a target adenosine present in an ESS of a target RNA molecule, wherein under physiological conditions the ESS would hybridize with an ESCS of an RNA molecule in a cell to form a double stranded RNA complex, the method comprising contacting the target RNA molecule with the AON of claim 1.

15. (canceled)

16. (canceled)

17. (canceled)

18. A method of treating or preventing a condition associated with erroneous or unwanted ADAR-mediated adenosine deamination, the method comprising administering an effective amount of the AON any of claim 1 to a patient in need thereof.

19. (canceled)

20. The method of claim 18, wherein the condition associated with erroneous or unwanted ADAR-mediated adenosine deamination is selected from the group consisting of :
- a viral infection, optionally a respiratory viral infection, optionally a respiratory viral infection caused by a coronavirus, optionally wherein the coronavirus is SARS-CoV-2;
- a metabolic disorder, optionally wherein the metabolic disorder is selected from the group consisting of hyperuricemia, obesity and cardiovascular disease;
- a mental disorder, optionally wherein the mental disorder is selected from the group consisting of depression, bipolar disorder, schizophrenia and suicide risk; and
- cancer, optionally wherein the cancer is selected from the group consisting of hepatocellular carcinoma, esophageal squamous cell carcinoma, non-small-cell lung cancer, colorectal cell carcinoma, cervical cancer, multiple myeloma, breast cancer, lung adenocarcinoma, prostate cancer, chronic myelogenous leukemia, head and neck squamous cell carcinoma, kidney renal papillary cell carcinoma, thyroid carcinoma and uterine corpus endometrial carcinoma.

21. The AON of claim 2, wherein the nucleotides surrounding the target adenosine consist of the adjacent base in the 3′ direction and the adjacent base in the 5′ direction.

22. The AON of claim 6, wherein the 2′-O ribosyl derivative is 2′-OMe or 2′-MOE.

23. The AON of claim 8, wherein the ADAR is human ADAR1 or human ADAR2.

24. The AON of claim 9, wherein the ESS and ESCS would hybridize under physiological conditions to form a stem-loop structure.
